(19) United States
(12) Patent Application Publication
(10) Pub. No.: US 2002/0015698 A1
(43) Pub. Date: Feb. 7, 2002

(54) Prophylaxis and therapy of diabetes mellitus I with the help of proteolytic enzymes

(76) Inventors: Karl Ransberger, Seeshaupt (DE); Gerhard Stauder, Geretsried (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 09/835,596

(22) Filed: Apr. 16, 2001

(30) Foreign Application Priority Data
Apr. 17, 2000 (DE) 100 18 980.6

(51) Int. Cl. A61K 38/43; A61K 38/46

(52) U.S. Cl. 424/94.65; 514/27

(57) ABSTRACT

The present invention relates to the use of proteolytic enzymes for the prophylaxis and/or therapy of type I diabetes mellitus. The proteolytic enzymes are preferably used at the prediabetic stage.

The present invention relates to the use of proteolytic enzymes for the prophylaxis and therapy of type I diabetes mellitus.

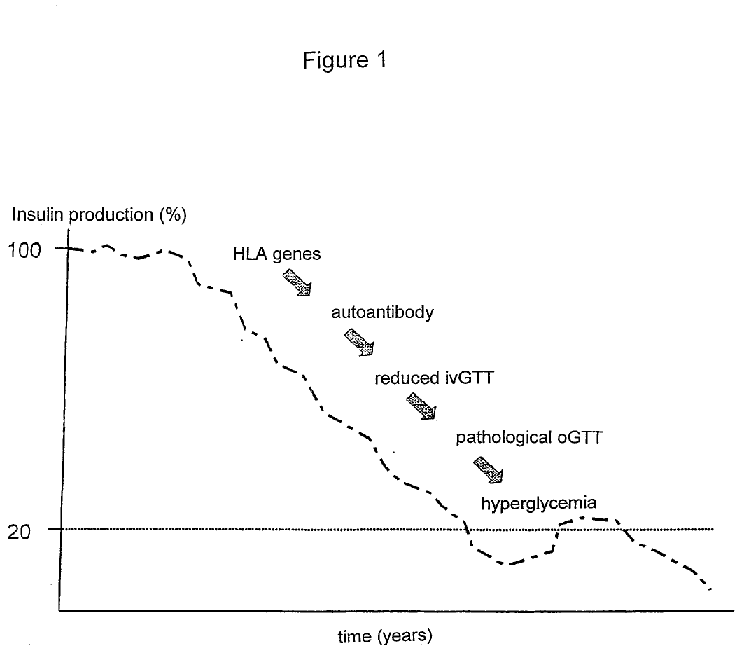

Type I diabetes mellitus is caused by an autoaggression of the immune system against insulin-producing cells in the islets of Langerhans. This process takes place for years without being noticed. It is only when about 70-80% of the insulin-producing β-cells are destroyed that the disease manifests itself through typical insulin deficiency symptoms, such as weight loss, increased thirst and urination (see FIG. 1). Already at the beginning of the nineties it was possible to isolate T-cells from the blood of freshly manifested type I diabetic persons who specifically react with an autoantigen from the membranes of insulin-producing cells. The 38 kD-large antigen was identified as “Imogen 38”.

The insulin deficiency caused by the destruction of the insulin-producing cells leads to a rise in the blood glucose level (hyperglycemia) and a consecutive secretion of glucose in the urine. The prevalence of type I diabetes mellitus in the normal population is about 0.1 to 0.3% in Europe, and a continuous increase in diabetes incidence was observed in many countries in the last years. Diabetes manifests itself around puberty in most cases, but in some patients a diabetes manifestation is only observed in later life.

Since the insulin-producing cells are destroyed in type I diabetes, clinical manifestation must be followed by an insulin therapy. A brief remission with a reduced insulin demand is observed in some patients after an initial insulin treatment. In a small percentage of the patients, an insulin therapy can even be dispensed with for some weeks. Afterwards the patients depend on insulin injections for the rest of their lives. Thanks to modern insulin therapies, the patients can lead a more or less normal life, but nevertheless have a reduced life expectancy. The known consecutive diseases of type I diabetes, such as neuro-, nephro-, retino- and angiopathy, can probably not be prevented entirely despite the consistent exploitation of today's therapeutic possibilities. This is in particular true for children and adolescents, with their specific growth- and puberty-related problems.

The reason for the destruction of the insulin-producing cells has so far not been known. The importance of genetic factors in the pathogenesis of type I diabetes mellitus follows from a concordance rate of 30-40% in identical twins. Especially genes from the HLA region (HLA-DR3 and/or 4) are dominating in type I diabetics. It is postulated that additional environmental factors participate in the pathogenesis of type I diabetes.

The objective of many studies in the past years was to detect the chronic inflammation of the insulin-producing cells prior to manifestation of the diabetes. For the early diagnosis in the so-called prediabetic state, various autoantibodies have turned out to be predictive. The islet cell antibodies (ICA) are the best evaluated autoantibodies for the early detection of type I diabetes. These are detected by means of indirect immunofluorescence on human frozen sections. Various autoantibodies are here concerned that react against specific islet proteins. Some autoantigens were identified in the past years. For instance, a large percentage of the islet cell antibodies consists of antibodies against glutamate decarboxylase (GAD). Further antibodies are directed against insulin (insulin autoantibody, IAA) and have a high predictive value, in particular in children. Furthermore, antibodies against the tyrosin phosphatases IA2 and IA2β take part.

In former efforts for preventing type I diabetes mellitus an immune intervention was e.g. tried out at the time of the manifestation of diabetes mellitus. The remission phase after manifestation of diabetes was enhanced and prolonged by treatment with cyclosporine A. However, despite a continued immunosuppressive therapy these remissions were lost after 2 to 3 years at the latest. Since cyclosporine A constitutes one of the most potent immunosuppressive substances, it must be assumed that the islet cell residue is too small at the time of the diabetes mellitus manifestation to achieve any healing of the diabetes. That is why efforts in diabetes prevention were directed at the prediabetic phase in recent years. This includes the “German Nicotinamide Intervention Study (DENIS)” in which the B vitamin nicotinamide was tested in siblings at the age of 3 to 12 years of children suffering from type I diabetes. However, an effect of nicotinamide on the pathogenesis of type I diabetes could not be detected. Within the scope of the “European Nicotinamide Intervention Study (ENDIT)” first relatives of type I diabetics are treated with nicotinamide up to the 40^{th} year of their lives. In further studies the early subcutaneous and oral insulin therapy was tested in subjects with islet cell antibodies and an impaired intravenous glucose tolerance.

The pathogenesis of type I diabetes mellitus can be regarded as a time-graded cascade from which the possibilities of early detection can also be inferred (see FIG. 1). On the basis of a genetic disposition which is localized in the region of the HLA-DR and -DQ genes, autoantibodies are observed. A reduction of the i.v.GTT (intravenous glucose tolerance test) is regarded as a further prediabetic stage. In the case of the pathological failure of the oGTT (oral glucose tolerance test) or the detection of hyperglycemia the criteria of a manifest diabetes mellitus are met.

Since with the presently available diagnostic means type I diabetes mellitus cannot be predicted with a 100% certainty, a treatment of children, adolescents and young adults of whom only a percentage will contract diabetes requires that use should only made of drugs with a side-effect profile that is as low as possible.

The present invention has been based on the technical problem to provide a further possibility for the prophylaxis or therapy of type I diabetes, wherein the side-effect profile should be as low as possible.

Said technical problem is solved according to the invention by the use of at least one proteolytic enzyme for the prophylaxis and/or therapy of type I diabetes mellitus.

As an indicator of the successful treatment of type I diabetes with hydrolytic enzymes, the change in the level of diabetes-specific autoantibodies, such as GAD, IA2, ICA, IAA, can be utilized. In many cases the use of hydrolytic enzymes slowed down the increase in said autoantibodies in comparison with untreated control patients, or the occurrence of the autoantibodies could be prevented in the subjects treated according to the invention, whereas some of the risk patients from the negative control group formed said autoantibodies in the course of time. As an alternative marker for the response of a patient to the treatment with hydrolytic enzymes the ratio of diabetes-promoting Th1 cytokines (IL12, TNF-α) and diabetes-inhibiting Th2 cytokines (IL4 and IL10) can be determined. Said factors can be determined by means of quantitative RT-PCR. This marker also shows the response of the patients treated according to the invention to the therapy with hydrolytic enzymes in that the ratio of said cytokines is shifted in favor of the diabetes-inhibiting cytokines.

Preferably the proteolytic enzyme is selected from trypsin, chymotrypsin, bromelain and papain and from combinations of said enzymes.

The enzymes used according to the invention can e.g. be isolated at low costs from the following raw material.

Bromelain is a proteolytically active enzyme from squeezed pineapple juice and can also be isolated from ripe fruits.

Papain is a proteolytic enzyme obtained from the latix of unripe fleshy fruits of the melon tree Carica papaya. Pure papain is a crystalline polypeptide with a molecular weight of 23,350 which consists of a chain of 212 amino-acid residues with 4 disulfide bridges. Sequence and spatial structure of the enzyme are known. Papain has many applications: Thanks to its protein-cleaving property it is used as a “meat tenderizer”, for clarifying beer, for making bread or hard biscuits, in leather preparation, in the textile industry, for boiling off silk and for preventing wool matting, in the tobacco industry for quality improvement, and for recovering silver from used photographic material, further in bacteriology for peptone isolation. In the medical field papain already serves to promote enzymatic digestion, it serves enzymatic wound cleaning or as an additive to cleaning agents for dental prostheses. For special purposes papain preparations are also offered bound to carriers such as plastic polymers or agarose. Papain has also been used as a catalyst for the synthesis of oligopeptides.

Trypsin is a proteolytic enzyme which is also formed in the pancreas. It belongs to the serine proteases. Crystalline trypsin has a molecular weight of about 23,300, it is soluble in water, but not in alcohol, it has an optimum effect at pH values of 7 to 9 and cleaves peptide chains specifically on the carboxyl terminal side of the basic amino-acid residues L-lysine and L-arginine. The spatial structure of trypsin, which consists of 223 amino acids, is known.

Chymotrypsin is also formed in the pancreas. It also belongs to the serine proteases. The best-studied α-chymotrypsin has a molecular weight of about 25,000 and comprises 245 amino acids.

In a further preferred embodiment flavonoids (flavone glycosides) are used as an additional active substance. This class of substances is wide-spread in the vegetable kingdom and can be isolated therefrom. Particularly preferred is rutoside (rutin).

In a particularly preferred embodiment 20-100 mg bromelain, 40-120 mg papain and 10-50 mg trypsin are used per dose unit, e.g. tablet.

In a further preferred embodiment use is made of 10-100 mg, particularly preferably 100 mg rutoside×3 H₂O per dose unit.

In a further preferred embodiment use is made of a combination of 90 mg bromelain, 120 mg papain and 100 mg rutoside×3 H₂O per dose unit.

A particularly preferred embodiment consists of the combination of 90 mg bromelain, 48 mg trypsin and 100 mg rutoside×3 H₂O per dose unit. This combination is e.g. sold under the name “Phlogenzym” by the company Mucos Pharma GmbH & Co. in Germany.

The dose unit may further contain all of the standard adjuvants or vehicles.

For instance lactose, magnesium stearate, stearic acid, talcum, methacrylic acid, copolymer type A, Shellack, Makrogel 6000, dibutyl phthalate, vanillin, titanium dioxide, white clay, polyindone, yellow wax and Carnauba wax are possible as adjuvants and vehicles.

In a further preferred embodiment the hydrolytic enzymes are used in patients in a prediabetic state which is characterized by the occurrence of antibodies against islet cells (ICA) or other autoimmune markers, e.g. antibodies against GAD65, tyrosin phosphatase IA2 or insulin, for the first time.

The following examples will explain the invention.

Determination of Glucose in Urea

Glucosuria can be determined by means of conventional test strips, such as Diabur-Test 5.000, Boehringer, Mannheim, Germany. If the test is positive, glucose is additionally determined in the blood. The glucose can e.g. be determined with the glucose analyzer Glucometer Elite, Bayer Diagnostics, Munich, Germany.

Determination of GAD-specific Autoantibodies

To this end a GAD radioimmunoassay was carried out according to the method of Wiest-Ladenburger, U. et al., Diabetes, Vol. 56, page 565 (1997). For this purpose recombinant human ³⁵S-GAD65 and ³⁵S-GAD67 are produced by means of a coupled transcription/translation system of Promega, Madison, Wis., USA. Expression plasmids containing the cDNAs of rGAD65 or rGAD67 were used as templates for transcription. Labeled proteins were separated from unincorporated ³⁵S-methionine on Sephadex G25 (Pharmacia, Uppsala, Sweden). 5 ul serum is incubated in duplicates with 15,000 cpm of radioactive protein at 4° C. overnight. Protein-A Sepharose is added and after 1 hour antibody-bound GAD is separated from unbound GAD by washing in membrane-bottom microtiter wells (Millipore, Eschborn, Germany). The counts per minute (cpm) were determined in a β-counter.

1. Use of at least one proteolytic enzyme for the prophylaxis and/or therapy of type I diabetes mellitus.

2. Use according to claim 1, characterized in that trypsin, chymotrypsin, bromelain or papain or a combination of several of said enzymes is used as the proteolytic enzyme.

3. Use according to claim 1 or 2, characterized in that a flavonoyl glycoside, preferably rutoside, is additionally used.

4. Use according to at least one of claims 1 to 3, characterized in that 20 to 100 mg bromelain, 40 to 120 mg papain and 10 to 50 mg trypsin are used per dose unit.

5. Use according to one or several of claims 1 to 4, characterized in that 90 mg bromelain, 120 mg papain and 100 mg rutoside are used per dose unit.

6. Use according to one or several of claims 1 to 4, characterized in that 90 mg bromelain, 48 mg trypsin and 100 mg rutoside are used per dose unit.

7. Use according to one or several of claims 1 to 6, characterized in that the prophylaxis of type I diabetes mellitus is carried out at the prediabetic stage.
